(19) United States
(12) Patent Application Publication
(10) Pub. No.: US 2022/0348607 A1
(43) Pub. Date: Nov. 3, 2022

(54) PROCESS FOR PREPARING (15ALPHA,16ALPHA,17BETA)-ESTRA-1,3,5(10)-TRIENE- 3,15,16,17-TETROL (ESTETROL) AND INTERMEDIATES OF SAID PROCESS

(71) Applicant: INDUSTRIALE CHIMICA S.R.L., Milano (IT)

(72) Inventors: Roberto LENNA, San Giorgio su Legnano (IT); Andrea FASANA, Nesso (IT); Riccardo LUCENTINI, Cislago (IT)

(73) Assignee: INDUSTRIALE CHIMICA S.R.L., Milano (IT)

(21) Appl. No.: 17/763,267

(22) PCT Filed: Sep. 25, 2020
(86) PCT No.: PCT/EP2020/076843

§ 371 Date: Mar. 24, 2022

(30) Foreign Application Priority Data
Sep. 27, 2019 (IT) 102019000017414
Nov. 22, 2019 (IT) 102019000021879

(51) Int. Cl. C07J 1/00

(52) U.S. Cl. CPC C07J 1/007

(57) ABSTRACT

The present invention relates to a process for preparing (15α,16α,17β)-Estra-1,3,5(10)-triene-3,15,16,17-tetrol, also known as Estetrol, having the formula shown below:

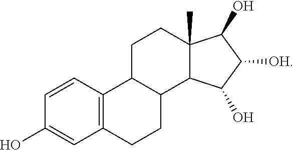 [SMILES: C[C@]12CCC3c4ccc(O)cc4CCC3C1[C@@H](O)[C@@H](O)[C@@H]2O]

### FIELD OF THE INVENTION

The present invention refers to the sector of processes for the synthesis of active ingredients for pharmaceutical use, and in particular to a process for preparing the compound on an industrial scale (15α,16α,17β)-estra-1,3,5(10)-triene-3,15,16,17-tetrol, also known as Estetrol, both in anhydrous and monohydrate form. The invention also relates to an intermediate of the process.

### BACKGROUND

The Estetrol compound is an active ingredient with pharmacological activity that makes it useful for Hormone Replacement Therapy (HRT), in female contraception, or in the therapy of autoimmune dysfunctions linked to hormonal imbalances.

The structural formula of Estetrol is reported below:

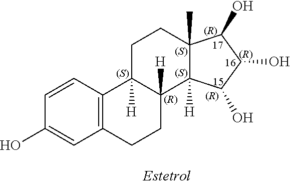

The positions 15, 16 and 17 of the steroidal skeleton (highlighted in the above reported formula) each bear one hydroxyl that, as indicated in the structural formula, have a defined spatial arrangement.

Estetrol is a natural product isolated from human urine and has been known for years; it has been described in the article “Synthesis of epimeric 15-hydroxyestriols, new and potential metabolites of estradiol”, J. Fishman et al., JOC Vol. 33, No. 8, August 1968, p. 3133-3135 (compound Ia of the figure on page 3133).

As far as the obtaining of Estetrol is concerned, the process obtainable from this article does not feature industrial applicability due to the low yield of the process.

Several patent applications have recently been published relating to new Estetrol synthesis processes but none of them avoids the formation of isomer 15β,16β,17β, having the structural formula shown below, from which Estetrol must be purified to be used in pharmaceutical preparations.

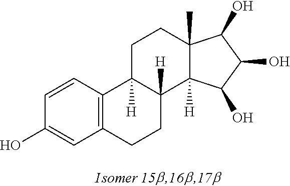

For example, application WO 2004/041839 A2 (page 6, lines 5-10) describes a process for obtaining Estetrol the purity of which can reach 99%, with the sum of the single impurities not exceeding 1%. Example 11 on page 28 describes an Estetrol with HPLC purity of 99.1% (HPLC-Ms) which however does not provide information on the content of the single impurities; the limit accepted by international guidelines for pharmaceutical substances is 0.1% for unknown ones and 0.15% for identified ones.

The content of impurities in an active ingredient (API) is an essential and non-derogable requirement to allow the use thereof in pharmaceutical preparations and is also a fundamental characteristic for defining an industrially applicable process. Any process, regardless of the yield, providing an API with an impurity content that does not respect the limits of the international guidelines is not an industrially useful process as the API, the result of the process, is not usable.

Subsequent applications relating to the production of Estetrol are, for example, WO 2012/164096 A1, WO 2013/050553 A1 and WO 2015/040051 A1.

In WO 2015/040051 A1 the ratio Estetrol/isomer 15β,16β,17β is equal to 99:1 in the examples 10 and 15, and equal to 98:2 in the examples 11 and 17. In these examples, however, no indication is given for lowering the content of isomer 15β,16β,17β to at least 0.15%. Even chromatographic purification (example 15) does not allow to obtain this result. In this document it is noted (page 9, lines 5-15) that the processes described in the discussed prior art (represented in the case of this document by applications WO 2012/164096 A1 and WO 2013/050553 A1) provide even higher and unacceptable amounts of isomer 15β,16β,17β.

It therefore appears clear that none of the described processes provides a solution to the limitation of the formation of the isomer 15β,16β,17β or a method of purification of Estetrol from said isomer.

### SUMMARY OF THE INVENTION

The object of the present invention is to provide an Estetrol synthesis process with a content of isomer 15β,16β,17β lower than 0.15%, without having to resort to purification techniques that are not industrially applicable.

In a first aspect thereof, the invention relates to a synthesis process of Estetrol which comprises the following steps:
A) oxidation of compound (17β)-3-(phenylmethoxy)-estra-1,3,5(10),15-tetraen-17-ol (intermediate 1) to give compound (17β)-3-(phenylmethoxy)-estra-1,3,5(10)-triene-15, 16,17 -triol (intermediate 2):

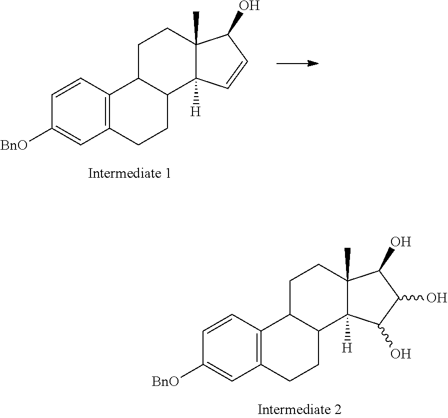

wherein Bn=benzyl, and in which the configuration of the carbon atoms 15 and 16 of the steroidal skeleton of intermediate 2 is not fixed;
B) acetylation of intermediate 2 to give compound (15α,16α,17β)-3-(phenylmethoxy)-estra-1, 3,5(10)-triene-15,16,17-triol triacetate (intermediate 3) passing through intermediate 3′ in which the configuration of the carbon atoms 15 and 16 of the steroidal skeleton is not fixed:

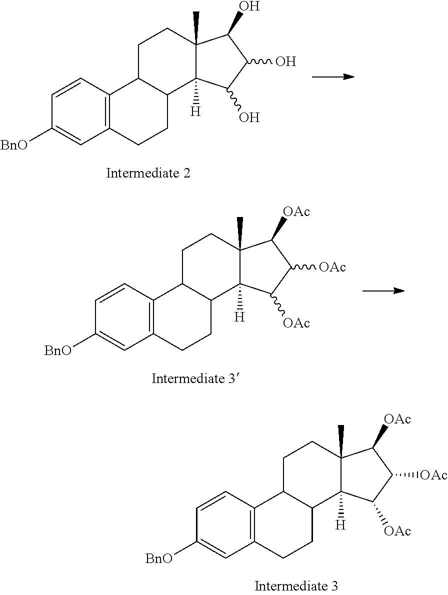 [SMILES: C[C@]12CCC3c4ccc(OCc5ccccc5)cc4CCC3[C@@H]1C(O)C(O)[C@@H]2O ; CC(=O)OC1C(OC(C)=O)[C@H](OC(C)=O)[C@@]2(C)CCC3c4ccc(OCc5ccccc5)cc4CCC3[C@H]12 ; CC(=O)O[C@@H]1[C@H](OC(C)=O)[C@H]2C3CCc4cc(OCc5ccccc5)ccc4C3CC[C@]2(C)[C@H]1OC(C)=O]

C) transformation of intermediate 3, passing through the compound (15α,16α,17β)-3-hydroxy-estra-1, 3,5(10)-triene-15,16,17-triol triacetate (intermediate 4), which is preferably not isolated, into Estetrol:

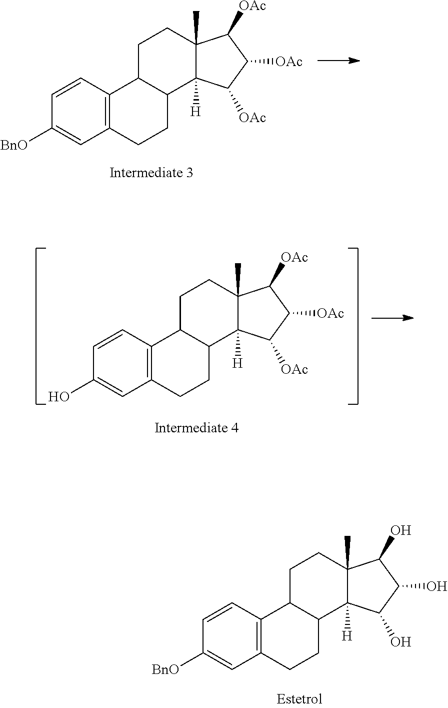

D) purification of Estetrol obtained in step C).

In an alternative embodiment, the process of the invention further comprises an additional step E), in which Estetrol produced in step D) is transformed into Estetrol monohydrate.

In a second aspect thereof, the invention relates to intermediate 3, (15α,16α,17β)-3-(phenylmethoxy)-estra-1, 3,5(10)-triene-15,16,17-triol triacetate:

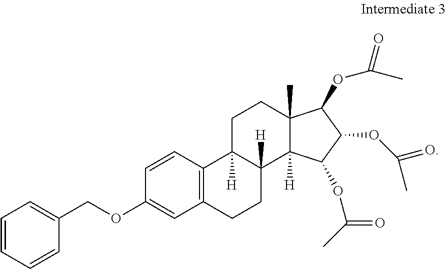

### BRIEF DESCRIPTION OF THE DRAWINGS

FIG. 1 shows the HPLC chromatogram of the Estetrol obtainable with the process of the invention.

FIG. 2 shows the HPLC chromatogram of Estetrol monohydrate obtainable with the process of the invention.

FIG. 3 shows the DRX diffractogram of the anhydrous and monohydrate Estetrol obtainable with the process of the invention.

FIG. 4 shows the DSC chromatogram of the anhydrous Estetrol obtainable with the process of the invention.

FIG. 5 shows the DSC chromatogram of Estetrol monohydrate obtainable with the process of the invention.

### DETAILED DESCRIPTION OF THE INVENTION

In a first aspect thereof, the invention relates to a synthesis process of Estetrol which comprises the steps defined above.

Step A) consists in the oxidation of the compound (17β)-3-(phenylmethoxy)-estra-1,3,5(10),15-tetraen-17-ol (intermediate 1) to give the compound (17β)-3-(phenylmethoxy)-estra-1, 3,5(10)-triene-15,16,17-triol (intermediate 2):

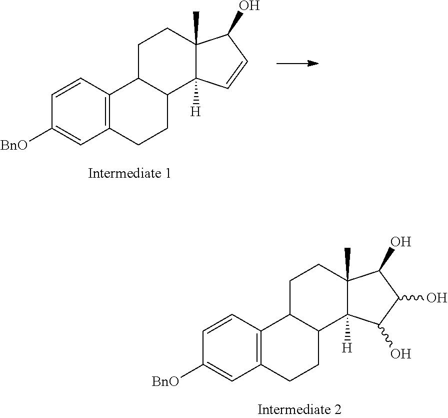

wherein Bn=benzyl, and in which the configuration of the carbon atoms 15 and 16 of the steroidal skeleton of intermediate 2 is not fixed.

The starting substrate of this step, intermediate 1, can be obtained as described in application WO 2004/041839 A2.

As oxidant in the reaction of step A) it is possible to use osmium tetroxide (OsO₄) supported on a polymer or, preferably, as such. An organic amine N-oxide, such as trimethylamine N-oxide dihydrate, is used as co-oxidant.

Since oxidation with OsO₄ is not stereoselective, intermediate 2 is obtained as a mixture of isomers with configuration 15α,16α,17β and 15β,16β,17β the isomer 15α,16α,17β is produced in preponderant amount together with a minority amount of isomer 15β,16β,17β.

The reaction is carried out in a solvent inert to osmium derivatives, such as tetrahydrofuran (THF), at a temperature between 35 and 60° C., preferably between 45 and 55° C., and for a time of at least 12 hours, preferably at least 16 hours.

The reaction product (intermediate 2) after work up is treated with a product sequestering metallic impurities in solution to eliminate the residual osmium content. These products, well known in chemistry, are generally based on a functionalized silica gel and commonly referred to in the sector by the term scavenger, which will be used in the rest of the text and the claims. The scavenger is preferably QuadraSil® MP.

The treatment with the scavenger can be carried out and can be repeated at each step of the process; it is preferably carried out in step A).

Step B) consists in the acetylation of intermediate 2 to give the compound (15α,16α,17β)-3-(phenylmethoxy)-estra-1, 3,5(10)-triene-15,16,17-triol triacetate (intermediate 3) passing through intermediate 3′ in which the configuration of the carbon atoms 15 and 16 of the steroidal skeleton is not fixed:

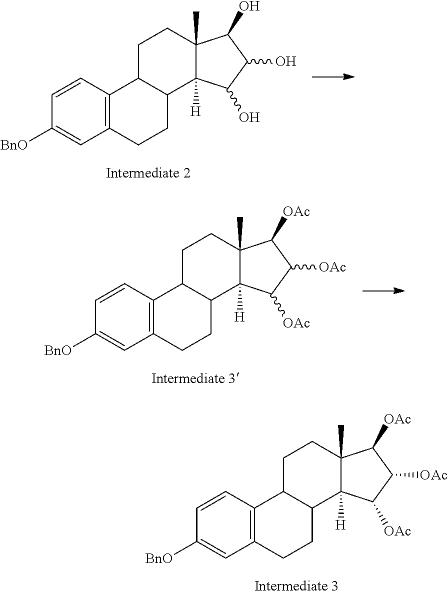 [SMILES: C[C@]12CCC3c4ccc(OCc5ccccc5)cc4CCC3[C@@H]1C(O)C(O)[C@@H]2O ; CC(=O)OC1C(OC(C)=O)[C@H](OC(C)=O)[C@@]2(C)CCC3c4ccc(OCc5ccccc5)cc4CCC3[C@H]12 ; CC(=O)O[C@@H]1[C@H](OC(C)=O)[C@H]2C3CCc4cc(OCc5ccccc5)ccc4C3CC[C@]2(C)[C@H]1OC(C)=O]

The intermediate 2, the starting substrate of the acetylation reaction, can be loaded into the reaction as a solid or, preferably, the solution obtained in step A) is directly used.

The direct result of the acetylation reaction of intermediate 2 is intermediate 3′, consisting of a mixture of isomers 15α,16α,17β and 15β,16β,17β; said mixture is then separated with a purification procedure which constitutes the second part of step B).

The exhaustive acetylation of step B) is carried out in a solvent compatible with the conditions of the reaction itself, such as, for example, isopropyl acetate, ethyl acetate, tetrahydrofuran, pyridine or toluene. The preferred solvent is pyridine.

For the reaction acetic anhydride is used as reactant, in the presence of an inorganic or organic base, of a catalyst and possibly of catalytic amounts of trifluoroacetic anhydride. Pyridine is preferably used as the organic base, and 4-dimethylaminopyridine as a catalyst.

The reaction temperature is between 5 and 40° C., preferably between 20 and 30° C.; the reaction time is at least 3 hours, preferably at least 4 hours.

The purification of the intermediate 3′, with elimination of the isomer 15β,16β,17β, is obtained with the sequence of operations described below:
B.1) a heat treatment which consists in refluxing the intermediate 3′ to be purified in a linear or branched C1-C6 aliphatic alcohol, for at least 10 minutes, preferably for at least 15 minutes;
B.2) stirring the slurry of intermediate 3′ to be purified in a linear or branched C1-C6 aliphatic alcohol, at a temperature between 15 and 35° C., preferably between 20 to 30° C. and even more preferably between 23 and 27° C. for a period of between 2 and 24 hours, preferably for a period of between 3 and 18 hours, even more preferably for a period of between 4 and 16 hours;
B.3) recovering the purified intermediate 3 by filtration.

The alcohol of the heat treatment (operation B.1) and of the slurry (operation B.2) can be the same or different; preferably the same alcohol is used, which preferably is methanol.

The intermediate 3 to be purified can be recovered by filtration after operation B.1) and resuspended in solvent to obtain the slurry of operation B.2), or the same solvent can be kept always operating in the same container.

The purification treatment of intermediate 3 can be repeated the number of times necessary to obtain the desired level of purity according to the initial content of the isomer 15β,16β,17β. Preferably the purification process is repeated for at least two times.

The inventors carried out a series of experimental tests by repeating three times the sequence of operations B.1, B.2 and B.3 on samples of intermediate 3′ containing 5% of isomer 15β,16β,17β; in the first of these tests, the operation B.2 of stirring the slurry was carried out three times for 16 h, in a second test three times for 8 h, and in a third test three times for 4 h; these tests confirmed that the procedure of the invention, comprising the operations B.1+B.2+B.3, led in all cases to a final product in which the content of isomer 15β,16β,17β was lower than 0.10%, and in some cases lower than 0.05%.

Step C) of the process of the invention consists of two consecutive reactions, a first debenzylation by catalytic hydrogenation of the intermediate 3 to form the intermediate 4, and then the hydrolysis of the acetates present in the intermediate 4, according to the scheme below:

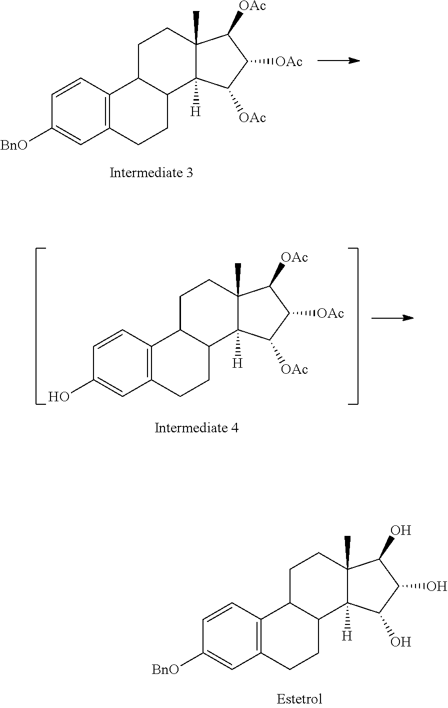

The order in which they are carried out is as indicated above. The catalytic debenzylation is performed first and then the hydrolysis of the acetates; the inversion of the order of reactions makes it difficult to complete debenzylation.

The intermediate 4 obtained from the first reaction can be isolated and then reacted again, but this intermediate is preferably kept dissolved in the solvent of the first reaction.

The conditions of debenzylation and hydrolysis are those known to chemists skilled in organic chemistry.

The first reaction, debenzylation, consists in a hydrogenation with gaseous hydrogen in the presence of a suitable catalyst. Preferred conditions for this reaction are:

use of palladium on charcoal (Pd/C) at 5% or preferably 10% by weight as a catalyst;

hydrogen pressure between 1 and 6 bar, preferably between 2 and 4 bar, even more preferably between 2.5 and 3.5 bar;

a linear or branched C1-C6 aliphatic alcohol, preferably methanol, as the reaction solvent;

reaction time of at least 16 hours, preferably at least 20 hours;

hydrogenation temperature between 30 and 60° C., preferably between 35 and 55° C., even more preferably between 40 and 50° C.

The second reaction consists in the hydrolysis of the acetates of intermediate 4, using bases. Preferred conditions for this reaction are:

use of sodium carbonate, potassium carbonate or lithium carbonate as a base; preferably potassium carbonate is used;

reaction time of at least 2 hours, preferably at least 4 hours;

reaction temperature between 10 and 40° C., preferably between 15 and 35° C., even more preferably between 20 and 30° C.

The solution containing the reaction product (Estetrol) can be treated with a functionalized silica gel-based scavenger to eliminate the residual content of palladium. The scavenger is preferably QuadraSil® MP.

Finally, the last step of the process of the invention, D), consists in the purification of Estetrol obtained in step C).

This step is carried out by hot-cold crystallization, according to methods known to the experts in organic chemistry.

The solvents used are tetrahydrofuran (THF), methanol and acetonitrile.

Also in this operation Estetrol can be treated with a functionalized silica gel-based scavenger, preferably QuadraSil® MP, to eliminate the residual content of palladium. The solvent in which to use the scavenger is selected from tetrahydrofuran (THF), methanol and acetonitrile; preferably tetrahydrofuran is used.

At the end of this operation, pure Estetrol is obtained in an “anhydrous” form, i.e. with a minimum residual water content, with a stoichiometric water/API ratio well below 1.

In an alternative embodiment, the invention is directed to the preparation of Estetrol in monohydrate form. In this embodiment, the process comprises a further step, E), which is carried out after step D) with the following sequence of operations:
E.1) dissolving pure Estetrol in anhydrous form in a water-miscible organic solvent such as acetone, methanol, ethanol, isopropanol, tetrahydrofuran, dimethylformamide or dimethylacetamide until complete solution; the preferred solvent is methanol;
E.2) mixing the solution of point E.1) with water, preferably pure water; preferably this operation is carried out by dripping the water onto the organic solution of Estetrol;
E.3) eliminating the organic solvent by distillation, preferably at reduced pressure;
E.4) maintaining the suspension under stirring, preferably for at least 15 minutes at a temperature ranging from 5 to 20° C.;
E.5) filtering and washing the solid; preferably the filtered solid is washed on the filter with water;
E.6) drying the solid for at least 5 hours at least 40° C. and reduced pressure, preferably for at least 6 hours at at least 45° C. and reduced pressure.

In a second aspect thereof, the invention relates to the purified intermediate 3, (15α,16α,17β)-3-(phenylmethoxy)-estra-1,3,5(10)-triene-15,16,17-triol triacetate, obtained during the process described above:

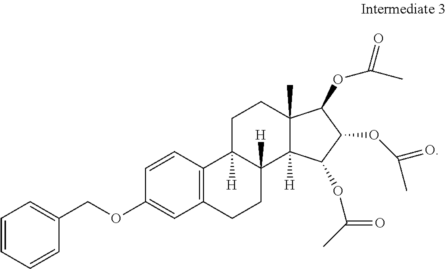

The invention will be further illustrated by the following examples.

EXPERIMENTAL INSTRUMENTS, METHODS AND CONDITIONS

NMR:

NMR spectrometer JEOL 400 YH (400 MHz); JEOL Delta software v5.1.1;

Spectra recorded in DMSO-d6.

MS:

Instrument: DSQ-trace Thermofisher

Sample introduction-direct exposure probe (dep)

Chemical ionization (CI) with methane

Methane pressure: 2.2 psi

Source temperature: 200° C.

HPLC:

Agilent Model 1260 Infinity chromatography system; UV Detector MODEL G1315C DAD VL+

Method HPLC 1:

Chromatographic conditions:

Column: Supelco ascentis express C18 250×4.6 mm, 5 μm

Flow: 1 ml/min

Detector: UV 280 nm

Injection volume: 5 μl

Temperature: 25° C.

Mobile phase A: water

Mobile phase B: acetonitrile

| TIME (min) | MOBILE PHASE A (v/v) | MOBILE PHASE B (v/v) |
|---|---|---|
| 0 | 80 | 20 |
| 0-5 | 80 | 20 |
| 5-45 | 20 | 80 |
| 45-55 | 20 | 80 |
| 55-56 | 80 | 20 |
| 56-66 | 80 | 20 |

Method HPLC 2:

Chromatographic conditions:

Column: Supelco discovery C18 150×4.6 mm, 5 μm

Flow: 1 ml/min

Detector: UV 280 nm

Injection volume: 25 μl

Temperature: 22° C.

Mobile phase A: 4.29 g/L solution of CH₃COONH₄ in water/methanol/acetonitrile 90/6/4

Mobile phase B: 38.6 g/L solution of CH₃COONH₄ in water/methanol/acetonitrile 10/54/36

| TIME (min) | MOBILE PHASE A (v/v) | MOBILE PHASE B (v/v) |
|---|---|---|
| 0 | 70 | 30 |
| 0-5 | 70 | 30 |
| 5-15 | 10 | 90 |
| 15-30 | 10 | 90 |
| 30-31 | 70 | 30 |
| 31-40 | 70 | 30 |

TLC:

MERCK: TLC silica gel 60 F254 Aluminum sheets 20×20 cm, code 1.0554.0001.

TLC Detector:

Cerium phosphomolybdate: 25 g of phosphomolybdic acid and 10 g of cerium (IV) sulfate are dissolved in 600 mL of H₂O. 60 mL of 98% H₂SO₄ are added it is and brought to 1 L with H₂O. The plate is impregnated with the solution and then heated until the products are detected.

XPRD:

The XRPD analysis was performed using a Bruker D2 Phaser (2nd edition) powder diffractometer operating in Bragg-Brentano geometry, equipped with a rotating multisampler and linear SSD type detector (Lynxeye). The X-ray source is an X-ray tube with a copper anode operated at 30 KV and 10 mA. For the analysis the X radiation having a wavelength corresponding to the average Kα of copper (λ=1.54184 Å) is used. The Kβ radiation is filtered through a special nickel filter.

“Zero background” silicon sample holders with a flat surface were used on which the sample was spread to form a thin layer. During the analysis the sample holder is rotated at a speed of 60 rpm.

Scanning is performed in the 4-40° 2Θ range with 0.016° 2Θ increments and an acquisition time of 1.0 s for each increment.

The diffractograms were processed using the Bruker DIFFRAC.EVA software.

DSC:

The DSC analysis was conducted in an inert atmosphere (nitrogen) using a Perkin Elmer Diamond DSC differential scanning calorimeter. Samples were prepared by weighing the powder into 40 μL aluminum crucibles, which were then sealed prior to analysis. The analysis was carried out in the temperature range 25-250° C. using a heating rate of 10° C./min

NOTES

The water used in the experimental descriptions is to be understood as pure water unless otherwise indicated.

The organic solvents used in the experimental descriptions are to be understood as of “technical” grade unless otherwise indicated.

The reagents and catalysts used in the experimental descriptions are to be understood as of commercial quality unless otherwise indicated.

The product QuadraSil® MP is available from Johnson Matthey.

### EXAMPLE 1

This example refers to step A) of the process of the invention, from intermediate 1 to intermediate 2.

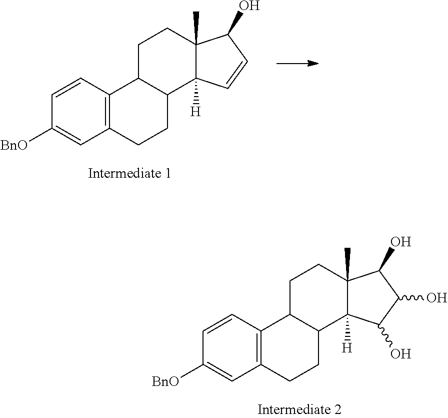 [SMILES: C[C@]12CCC3c4ccc(OCc5ccccc5)cc4CCC3[C@@H]1C=C[C@@H]2O ; C[C@]12CCC3c4ccc(OCc5ccccc5)cc4CCC3[C@@H]1C(O)C(O)[C@@H]2O]

In a flask under nitrogen, 32.4 g of intermediate 1 (89.87 mmol, 1 eq) and 356 mL of tetrahydrofuran were loaded. 0.324 g of osmium tetroxide (1.28 mmol, 1% by weight) and 17.9 g of trimethylamine N-oxide dihydrate (161.26 mmol, 1.8 eq) were added in this order to the solution. The system was heated to 50° C. and kept under stirring for 16 hours.

The reaction was controlled by TLC analysis under the following conditions: TLC plate: silica gel on alumina; starting substrate (intermediate 1) dissolved in dichloromethane; reaction mixture diluted in dichloromethane; eluent: ethyl acetate (EtOAc); detector: cerium phosphomolybdate.

At the end of the reaction, the solution was cooled to 25° C. and a solution of sodium metabisulphite (18.3 g) in water (162 mL) was dripped. The solvent was concentrated at reduced pressure and 193 mL of isopropyl acetate and 290 mL of 1M hydrochloric acid were added to the residue.

1.6 g of charcoal and 1.6 g of dicalite were added to the biphasic system and it was kept under stirring at 25° C. for 15 minutes. The suspension was first filtered on a dicalite layer and then on a Millipore filter (0.22 μm). The phases were separated and the aqueous phase was extracted with 160 mL of isopropyl acetate. 1.12 g of QuadraSil® MP were added to the organic phase and the system was kept under stirring at 25° C. for 16 hours. The suspension was filtered on a Millipore filter (0.22 μm) washing with 32 mL of isopropyl acetate.

The solution thus obtained was used as such in the subsequent reaction.

### EXAMPLE 2

This example refers to step B) of the process of the invention.

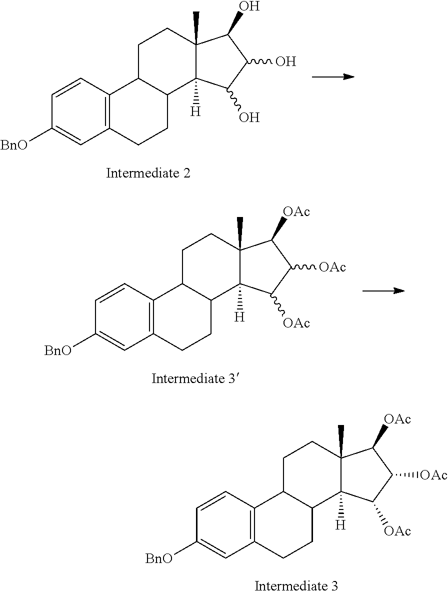 [SMILES: C[C@]12CCC3c4ccc(OCc5ccccc5)cc4CCC3[C@@H]1C(O)C(O)[C@@H]2O ; CC(=O)OC1C(OC(C)=O)[C@H](OC(C)=O)[C@@]2(C)CCC3c4ccc(OCc5ccccc5)cc4CCC3[C@H]12 ; CC(=O)O[C@@H]1[C@H](OC(C)=O)[C@H]2C3CCc4cc(OCc5ccccc5)ccc4C3CC[C@]2(C)[C@H]1OC(C)=O]

The solution of intermediate 2 obtained as described in the previous example was concentrated at reduced pressure to a residual volume of 50 mL.

228 ml of pyridine were added and the residual isopropyl acetate was distilled off at reduced pressure. 0.877 g of 4-dimethylaminopyridine (7.19 mmol, 0.08 eq) were added to the solution and then 29.45 mL of acetic anhydride (312 mmol, 3.47 eq) were dripped while keeping the temperature below 30° C. The solution was kept under stirring at 25° C. for 4 hours.

The reaction was controlled by TLC analysis, under the following conditions: TLC plate: silica gel on alumina; starting substrate (intermediate 2) dissolved in dichloromethane; reaction mixture quenched in 1M HCl and extracted with EtOAc, the organic phase was deposited; eluent: EtOAc; detector: cerium phosphomolybdate.

The reaction mixture was concentrated at reduced pressure to a residual volume of 85 mL and 250 mL of isopropyl acetate and 125 mL of water were added. 55 mL of 37% hydrochloric acid were added to the biphasic system, while keeping the temperature below 30° C. (final pH of the aqueous phase=1).

The phases were separated and the organic phase was washed twice with saturated sodium is bicarbonate solution (2×90 mL) and subsequently with saturated sodium chloride solution (90 mL).

The organic phase was concentrated at reduced pressure to an oily residue. 100 mL of methanol were added and the mixture was concentrated again at reduced pressure to a paste. 210 mL of methanol were added and the system was refluxed for 15 minutes. The suspension was cooled to 25° C. and kept under stirring for 16 hours. The solid was filtered on büchner washing with 35 mL methanol. The solid was dried at reduced pressure at 45° C. for 3 hours.

28.4 g of solid which constitutes the intermediate 3′ were obtained; with an HPLC analysis (method 1) a content of isomer 15β,16β,17β=1.6% was detected.

The solid (28 g) was dissolved with 168 mL of methanol and the system was refluxed for 15 minutes. The suspension was cooled to 25° C. and kept under stirring for 16 hours. The solid was filtered on büchner washing with 28 mL of methanol, and then dried at reduced pressure at 45° C. for 3 hours. 24 g of product were obtained (HPLC, method 1): isomer 15β,16β,17β=0.18%).

The solid (23.5 g) was dissolved with 140 mL of methanol and the system was refluxed for 15 minutes. The suspension was cooled to 25° C. and kept under stirring for 16 hours. The solid was filtered on büchner washing with 23 mL of methanol and dried under vacuum at 45° C. for 3 hours.

22.1 g of intermediate 3 (almost white solid) were obtained.

HPLC purity (method 1): 97.5%, isomer 15β,16β,17β=0.07%.

¹H-NMR (400 MHz, DMSO-d₆): δ 7.39-7.26 (m, 5H); 7.12 (d, 1H, J=9.2 Hz); 6.72-6.67 (m, 2H); 5.22-5.18 (t, 1H, J=7.4 Hz); 5.04-4.99 (m, 3H); 4.84 (d, 1H, J=6.4 Hz); 2.74-2.70 (m, 2H); 2.25-2.20 (m, 2H); 1.99-1.97 (2s, 9H); 1.7-1.2 (m, 7H); 0.85 (s, 3H).

Mass (CI): m/z=521 [M⁺+1].

### EXAMPLE 3

This example refers to the implementation of step C) of the process of the invention.

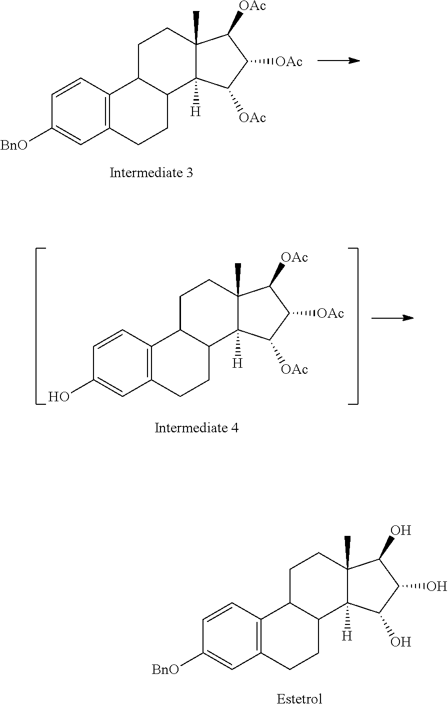

21.6 g of intermediate 3 obtained as described in the previous example and 154 mL of tetrahydrofuran were loaded into a flask.

2.2 g of QuadraSil® MP were added to the solution and the system was kept under stirring at 25° C. for 16 hours. The suspension was filtered on a Millipore filter (0.22 μm) washing with 22 ml of tetrahydrofuran. The solvent was concentrated at reduced pressure to a paste.

The residue was dissolved with 650 ml of methanol and loaded into a hydrogenation reactor 2.05 g of 10% palladium on charcoal were added to the suspension and hydrogenation was carried out at 45° C. and 3 bar for 22 hours.

The reaction was controlled by TLC analysis under the following conditions: TLC plate: silica gel on alumina; starting substrate (intermediate 3) dissolved in dichloromethane; reaction mixture diluted with methanol; eluent: heptane/EtOAc 1/1; detector: cerium phosphomolybdate. At the end of the reaction the system was filtered on a layer of dicalite (30 g) washing with methanol (120 mL).

The solvent was concentrated at reduced pressure to a residual volume of 430 mL and 5.16 g of potassium carbonate were added. The mixture was kept under stirring at 25° C. for 4 hours. The reaction was controlled by TLC analysis under the following conditions: TLC plate: silica gel on alumina; intermediate product 4 dissolved in dichloromethane; reaction mixture quenched in 1M HCl and extracted with EtOAc, the organic phase was deposited; eluent: heptane/EtOAc 1/1; detector: cerium phosphomolybdate. The suspension was filtered on a Millipore filter (0.22 μm) washing with methanol (20 mL).

The solution was concentrated at reduced pressure to a residual volume of 54 mL, 162 mL of water were added and the residual methanol was removed at reduced pressure.

The suspension obtained was neutralized with 40 mL of 1M hydrochloric acid and cooled to 10° C. while stirring for 30 minutes. The solid was filtered on büchner washing with water and dried at reduced pressure at 50° C. for 6 hours.

13 g of raw Estetrol (white solid) were obtained.

### EXAMPLE 4

This example refers to the implementation of step D) of the process of the invention.

The raw Estetrol, obtained as described in the previous example, was dissolved in 91 mL of tetrahydrofuran. 0.4 g of QuadraSil® MP were added to the solution and the system was kept under stirring at 25° C. for 16 hours. The suspension was filtered on Millipore (0.22 μm) washing with 25 ml of tetrahydrofuran. The solvent was removed at reduced pressure and 130 mL of acetonitrile and 104 mL of methanol were added. The system was kept under stirring at 25° C. until complete dissolution.

The solution was concentrated at reduced pressure to a residual volume of 130 mL and 104 mL of acetonitrile were added. The system was concentrated again at reduced pressure to a residual volume of 130 mL and 104 mL of acetonitrile were added.

The system was concentrated at reduced pressure to a residual volume of 130 mL and kept under stirring at 25° C. for 3 hours. The suspension was cooled to 5° C. and kept under stirring for 1 hour. The solid was filtered on büchner washing with cold acetonitrile, and dried at reduced pressure for 3 hours at 45° C.

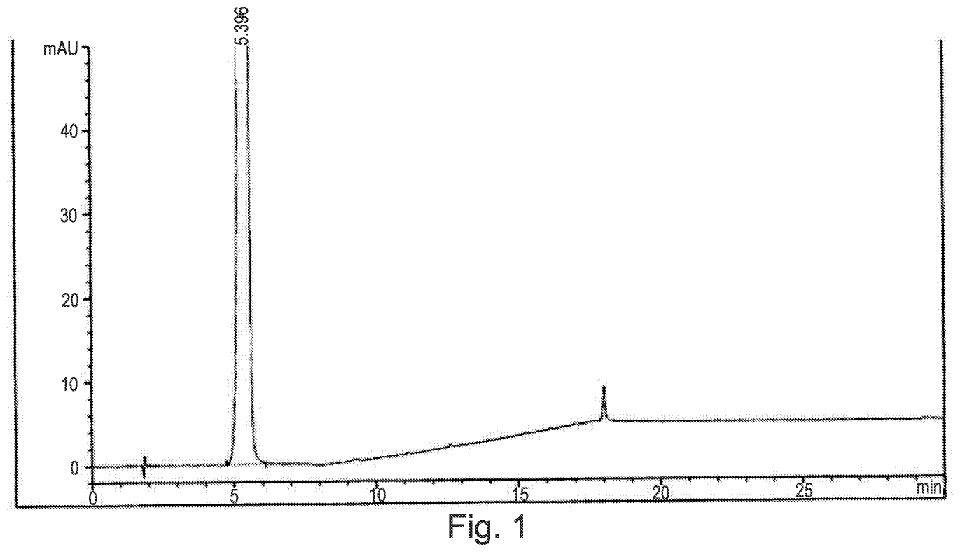

10.5 g of product were obtained, which was analysed by HPLC (method HPLC 2). The results of the test are shown in FIG. 1: the product was found to be Estetrol of HPLC purity=99.91%, with the isomer 15β,16β,17β not detectable (the peak with retention time of about 18′ is not attributable to the product but to the chromatographic elution itself).

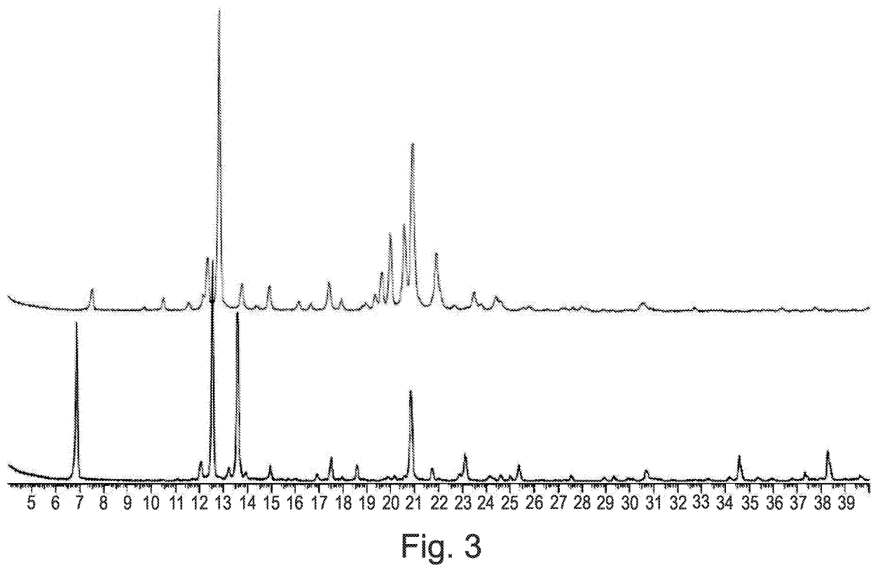

A sample of the product was subjected to XPRD analysis; the result of the test is the diffractogram shown in the upper part of FIG. 3. The table below shows the positions (as angle values 2Θ±0.2°) and the relative intensities of the main peaks of the diffractogram:

| Diffraction angle (2θ) | Relative intensity (%) |
|---|---|
| 7.49 ± 0.2 | 6.9 |
| 12.177 ± 0.2 | 4.4 |
| 12.324 ± 0.2 | 16.8 |
| 12.819 ± 0.2 | 100.0 |
| 13.769 ± 0.2 | 8.4 |
| 14.919 ± 0.2 | 7.7 |
| 17.408 ± 0.2 | 9.5 |
| 19.357 ± 0.2 | 4.7 |
| 19.618 ± 0.2 | 12.1 |
| 19.976 ± 0.2 | 25.3 |
| 20.57 ± 0.2 | 26.8 |
| 20.911 ± 0.2 | 55.4 |
| 21.909 ± 0.2 | 18.6 |
| 23.487 ± 0.2 | 5.6 |
| 24.41 ± 0.2 | 4.3 |

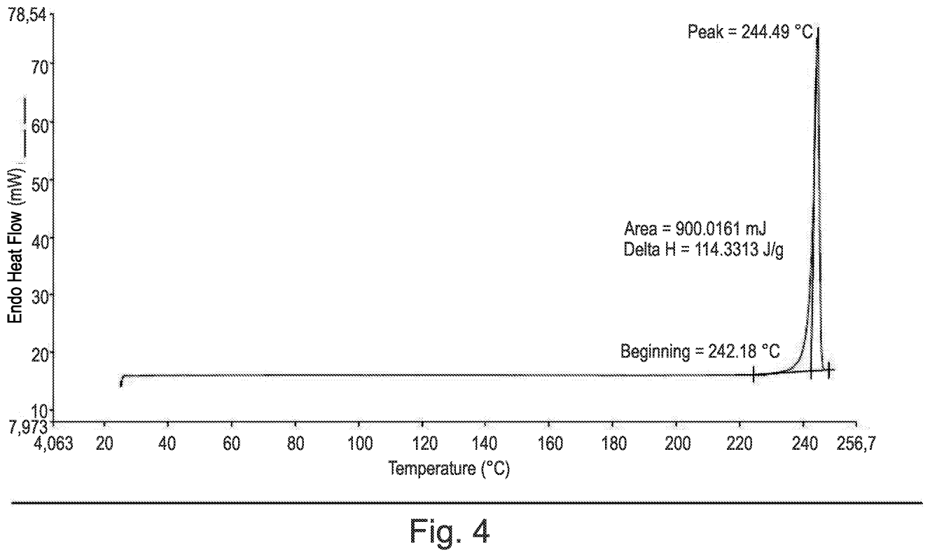

Another sample weighing 8 mg of the product obtained was subjected to DSC test; the result of the test is shown in FIG. 4, which shows that the product has a melting T of about 244.5° C.

### EXAMPLE 5

This example refers to the implementation of step E) of the process of the invention. 8 g of Estetrol obtained in Example 4 were dissolved in 96 mL of methanol and 240 ml of water were dripped into the solution thus prepared. The system was concentrated at reduced pressure until the methanol was completely removed. The suspension was kept under stirring at 15° C. for 30 minutes and the solid filtered on büchner washing with 56 mL of water.

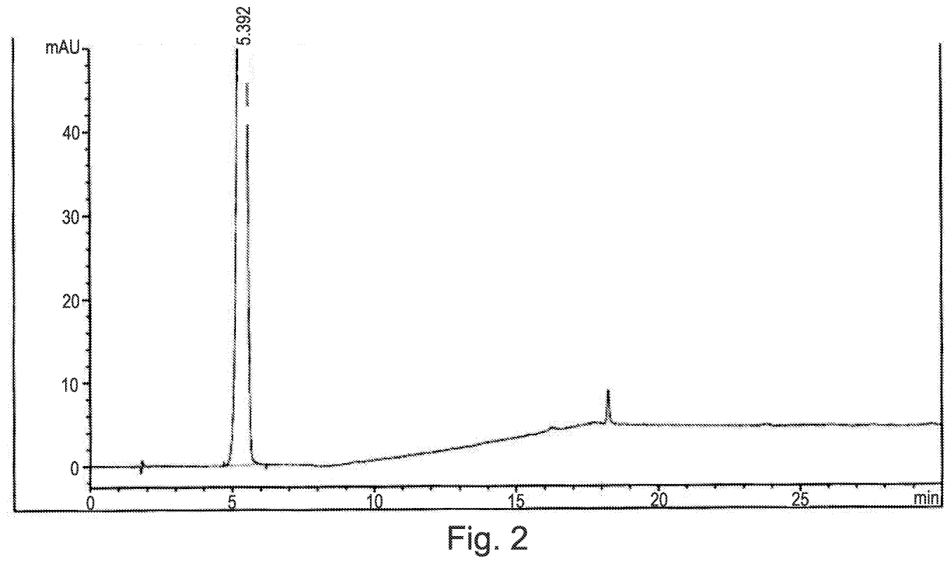

The solid was dried at reduced pressure at 45° C. for 6 hours. 8.3 g of Estetrol monohydrate (white solid) were obtained and analysed by HPLC (method 2). The results of the test are shown in FIG. 2: the product was found to be Estetrol monohydrate of HPLC purity=100% (the peak at a retention time of about 18′ is not attributable to the product but to the chromatographic is elution itself).

A sample of the product was subjected to XPRD analysis; the result of the test is the diffractogram shown in the lower part of FIG. 3. The table below shows the positions (as angle values 2Θ±0.2°) and the relative intensities of the main peaks of the diffractogram:

| Diffraction angle (2θ) | Relative intensity (%) |
|---|---|
| 6.846 ± 0.2 | 71.8 |
| 12.058 ± 0.2 | 8.3 |
| 12.533 ± 0.2 | 100.0 |
| 13.226 ± 0.2 | 4.9 |
| 13.586 ± 0.2 | 76.9 |
| 14.953 ± 0.2 | 6.1 |
| 17.501 ± 0.2 | 10.4 |
| 18.589 ± 0.2 | 6.8 |
| 20.845 ± 0.2 | 40.4 |
| 21.728 ± 0.2 | 5.0 |
| 23.109 ± 0.2 | 11.3 |
| 25.363 ± 0.2 | 6.7 |
| 30.698 ± 0.2 | 4.2 |
| 34.609 ± 0.2 | 7.6 |
| 38.320 ± 0.2 | 9.2 |

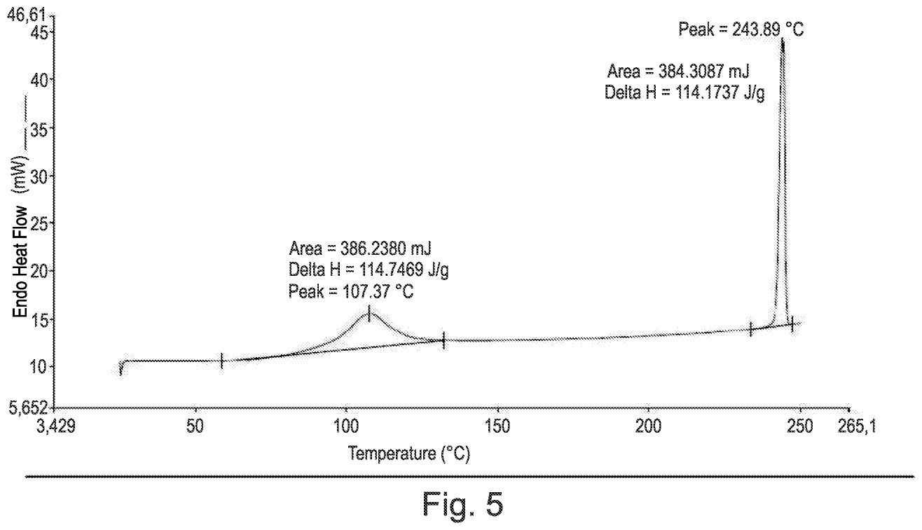

Another sample weighing 3.4 mg of the product obtained was subjected to DSC test; the result of the test is shown in FIG. 5, which shows a first widened peak with a maximum at about 107.4° C., attributed to the dehydration of Estetrol monohydrate, and a second peak at about 244° C., i.e. at a temperature essentially corresponding to the melting temperature of Estetrol found in the test of FIG. 4.

¹H-NMR (400MHz, DMSO-d₆): δ 9.0 (s, 1H); 7.05 (d, 1H, J=8.4 Hz); 6.51-6.48 (m, 1H); 6.27 (d, 1H, J=2.4 Hz); 4.86-4.85 (d, 1H, J=4.8 Hz); 4.61-4.59 (d, 1H, J =5.6 Hz); 4.27-4.26 (d, 1H, J=6 Hz); 3.72-3.66 (m, 2H); 3.26-3.24 (t, 1H, J=5.6 Hz); 2.72-2.68 (m, 2H); 2.22-2.18 (m, 2H); 2.1-2.05 (m, 1H); 1.76-1.73 (d, 1H, 12Hz); 1.4-1.03 (m, 5H); 0.66 (s, 3H).

Mass (CI): m/z=305 [M⁺+1].

1. Process for the synthesis of Estetrol, (15α,16α,17β)-estra-1,3,5(10)-triene-3, 15,16,17-tetrol, comprising the following steps:
A) oxidation of compound (17β)-3-(phenylmethoxy)-estra-1,3,5(10),15-tetraen-17-ol (intermediate 1) to give the compound (17β)-3-(phenylmethoxy)-estra-1,3,5(10)-triene-15, 16,17-triol (intermediate 2): 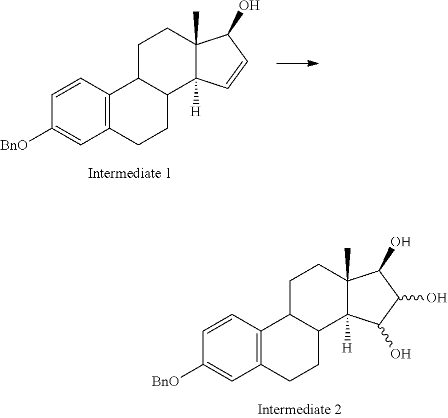
wherein Bn=benzyl, and in which the configuration of the carbon atoms 15 and 16 of the steroidal skeleton is not fixed;
B) acetylation of intermediate 2 to give compound (15α,16α,17β)-3-(phenylmethoxy)-estra-1, 3,5(10)-triene-15,16,17-triol triacetate (intermediate 3) passing through intermediate 3′ in which the configuration of the carbon atoms 15 and 16 of the steroidal skeleton is not fixed: 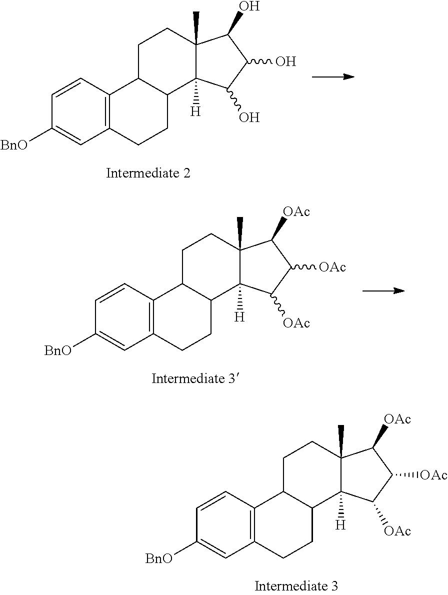 [SMILES: C[C@]12CCC3c4ccc(OCc5ccccc5)cc4CCC3[C@@H]1C(O)C(O)[C@@H]2O ; CC(=O)OC1C(OC(C)=O)[C@H](OC(C)=O)[C@@]2(C)CCC3c4ccc(OCc5ccccc5)cc4CCC3[C@H]12 ; CC(=O)O[C@@H]1[C@H](OC(C)=O)[C@H]2C3CCc4cc(OCc5ccccc5)ccc4C3CC[C@]2(C)[C@H]1OC(C)=O]
C) transformation of intermediate 3, passing through the compound (15α,16α,17β)-3-hydroxy-estra-1, 3,5(10)-triene-15,16,17-triol triacetate (intermediate 4), which is preferably not isolated, into Estetrol: 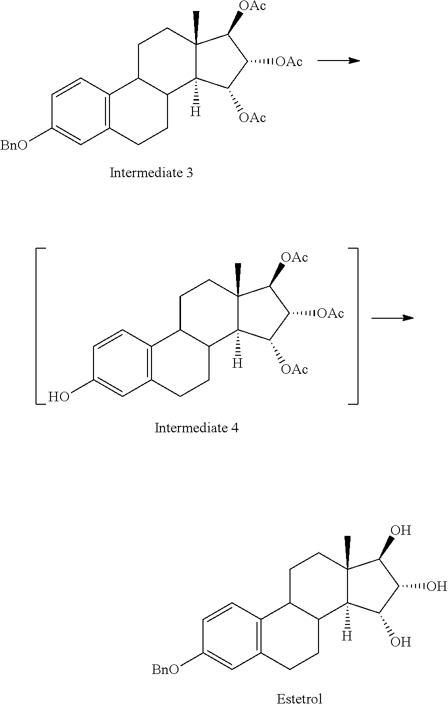
D) purification of Estetrol obtained in step C).

2. The process according to claim 1, wherein step A) is carried out using osmium tetroxide (OsO₄) as such or supported on a polymer as an oxidant and an organic amine N-oxide as a co-oxidant, operating in a solvent inert to the derivatives of osmium, at a temperature between 35 and 60° C., and for a time of at least 12 hours.

3. The process according to claim 2, in which step A) is carried out using osmium tetroxide (OsO₄) as such as an oxidant and trimethylamine N-oxide dihydrate as co-oxidant, operating in tetrahydrofuran (THF) as a solvent, at a temperature between 45 and 55° C., and for a time of at least 16 hours.

4. The process according to claim 1, in which in step B) the exhaustive acetylating reaction from intermediate 2 to intermediate 3′ is carried out using acetic anhydride as a reactant in a solvent selected from isopropyl acetate, ethyl acetate, tetrahydrofuran, pyridine and toluene, in the presence of an inorganic or organic base, of a catalyst and possibly of catalytic amounts of trifluoroacetic anhydride, and operating at a temperature between 5 and 40° C. for a time of at least 3 hours.

5. The process according to claim 4, wherein the exhaustive acetylating reaction from intermediate 2 to intermediate 3′ of step B) is carried out in pyridine as a solvent, 4-dimethylaminopyridine as a catalyst, operating at a temperature between 20 and 30° C. for a time of at least 4 hours.

6. The process according to claim 1, in which in step B) the purification of intermediate 3′ to give the intermediate 3 is carried out with the following sequence of operations:
B.1) refluxing intermediate 3′ to be purified in a linear or branched C1-C6 aliphatic alcohol, for at least 10 minutes, preferably for at least 15 minutes;
B.2) stirring the slurry of intermediate 3′ to be purified in a linear or branched C1-C6 aliphatic alcohol, at a temperature between 15 and 35° C., preferably between 20 to 30° C. and even more preferably between 23 and 27° C. for a period of between 2 and 24 hours, preferably for a period of between 3 and 18 hours, even more preferably for a period of between 4 and 16 hours;
B.3) recovering the purified intermediate 3 by filtration.

7. The process according to claim 1, in which the debenzylation reaction of step C), from intermediate 3 to intermediate 4, is carried out by hydrogenation with gaseous hydrogen in the presence of a catalyst.

8. The process according to claim 7, wherein said debenzylation reaction is carried out under the following conditions:
use of palladium on charcoal (Pd/C) at 5% or 10% by weight as a catalyst;
hydrogen pressure between 1 and 6 bar;
a linear or branched C1-C6 aliphatic alcohol, as a reaction solvent;
reaction time of at least 16 hours;
hydrogenation temperature between 30 and 60° C.

9. The process according to claim 1, in which the hydrolysis reaction of step C), from intermediate 4 to Estetrol, is carried out under the following conditions:
use of sodium carbonate, potassium carbonate or lithium carbonate as a base;
reaction time of at least 2 hours;
reaction temperature between 10 and 40° C.

10. The process according to claim 1, in which step D) is carried out by hot-cold crystallization, in a solvent selected from tetrahydrofuran, methanol and acetonitrile.

11. The process according to claims 1, further comprising an additional step E) in which Estetrol produced in step D) is transformed into Estetrol monohydrate according to the following sequence of operations:
E.1) dissolving pure Estetrol in anhydrous form in a water-miscible organic solvent such as acetone, methanol, ethanol, isopropanol, tetrahydrofuran, dimethylformamide or dimethylacetamide until complete solution;
E.2) mixing the solution of point E.1) with water, preferably pure water;
E.3) eliminating the organic solvent by distillation, preferably at reduced pressure;
E.4) maintaining the suspension under stirring, preferably for at least 15 minutes at a temperature ranging from 5 to 20° C.;
E.5) filtering and washing the solid;
E.6) drying the solid for at least 5 hours at at least 40° C. and reduced pressure.

12. Compound (15α,16α,17β)-3-(phenylmethoxy)-estra-1,3,5(10)-triene-15,16,17-triol triacetate:

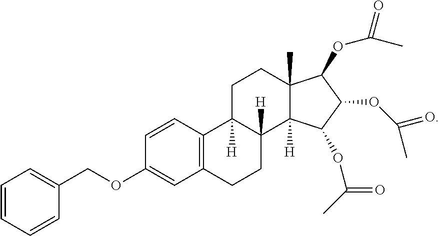

13. Process for the synthesis of Estetrol, (15α,16α,17β)-estra-1,3,5(10)-triene-3, 15,16,17-tetrol, which comprises the intermediate (15α,16α,17β)-3-(phenylmethoxy)-estra-1, 3,5(10)-triene-15,16,17-triol triacetate as necessary intermediate compound.
